(19) United States
(12) Patent Application Publication
(10) Pub. No.: US 2015/0360009 A1
(43) Pub. Date: Dec. 17, 2015

(54) MULTIFUNCTIONAL OCCLUSION CROSSOVER DEVICE

(71) Applicants: Boston Scientific SciMed, Inc., Maple Grove, MN (US); Mayo Foundation for Medical Education and Research, Rochester, MN (US)

(72) Inventors: Roger McGowan, Otsego, MN (US); Kim Davis, Minneapolis, MN (US); Rachel Japuntich, Roseville, MN (US); Umang Anand, Maple Grove, MN (US); Raghav Goel, Maple Gove, MN (US); Peter M. Pollak, Rochester, MN (US); Douglas Pennington, Stillwater, MN (US); Atta Behfar, Rochester, MN (US); Charles J. Bruce, Rochester, MN (US); Paul A. Friedman, Rochester, MN (US); Bruce D. Johnson, Rochester, MN (US); Lyle D. Joyce, Rochester, MN (US); Lyle J. Olson, Rochester, MN (US)

(21) Appl. No.: 14/738,208

(22) Filed: Jun. 12, 2015

Related U.S. Application Data
(60) Provisional application No. 62/011,742, filed on Jun. 13, 2014.

(51) Int. Cl. A61M 27/00; A61M 39/22; A61M 1/36; A61M 25/10; A61M 5/00

(52) U.S. Cl. CPC A61M 27/002; A61M 25/10; A61M 5/007; A61M 1/3666; A61M 39/22; A61M 2025/1052

(57) ABSTRACT

Multifunctional occlusion crossover devices include features to improve the crossover balloon occlusion technique used during transcatheter aortic valve replacement procedures, for example. The devices include a compliant balloon incorporated with a highly flexible and lubricous sheath system. The balloon is capable of safely occluding from the common iliac artery and down to an access site in the femoral artery. The devices can simplify the CBOT technique and replace multiple devices currently used for the procedure.

### CLAIM OF PRIORITY

This application claims priority to U.S. Provisional Application Ser. No. 62/011,742, filed on Jun. 13, 2014, the entire contents of which are hereby incorporated by reference.

### BACKGROUND

1. Technical Field

This document relates to devices and methods that may improve medical procedural efficacy and efficiency. For example, this document relates to multifunctional occlusion crossover devices with features that can improve the efficiency and may improve the efficacy of the crossover balloon occlusion technique used for percutaneous artery access.

2. Background Information

Transcatheter aortic valve replacement (TAVR) is a beneficial technique for the treatment of patients with symptomatic, severe aortic stenosis who are at high surgical risk. However, one current technical challenge associated with the TAVR technique is that it is performed using a large bore delivery sheath system typically requiring an arteriotomy of greater than 14 Fr. Vascular access management can be a major challenge in TAVR due to the large introducer sheaths required. For example, in some cases the large bore systems can injure the iliac and/or femoral vessels causing dissections and perforations. Significant bleeding from such complications may occur in as many as 10% of large bore access cases.

A technique currently used to improve safety and reduce complications of the large bore devices is known as the “crossover balloon occlusion technique” (CBOT) or simply the “crossover technique.” The crossover technique utilizes a femoral artery access site contralateral to the large bore access site with a wire up and over the iliac arch to maintain wire access to the true lumen and to provide some proximal blood flow control. The crossover technique can be used to manage the potentially catastrophic complications of iliac or femoral rupture without immediate hemodynamic collapse or massive hemorrhage.

### SUMMARY

This document provides devices and methods that may improve medical procedural efficacy and efficiency. For example, this document provides multifunctional occlusion crossover devices (also referred to herein as “CBOT devices”) with features that can improve the efficiency and may improve the efficacy of the crossover technique used for percutaneous artery access.

In one implementation, a multifunctional occlusion crossover device includes an elongate sheath defining a first lumen and a second lumen, a balloon attached to a distal end portion of the sheath, a hemostatic valve attached to a proximal end portion of the sheath, a one-way valve coupled to the device and configured such that the first lumen is fluidly accessible via the one-way valve, and a port coupled to the device and configured such that the second lumen is fluidly accessible via the port. The elongate sheath is diametrically expandable. The balloon is in fluid communication with the second lumen. A seal of the hemostatic valve defines a proximal end of the first lumen such that the first lumen is accessible via the seal.

Such a multifunctional occlusion crossover device may optionally include one or more of the following features. The elongate sheath may include two or more portions having differing mechanical properties. The mechanical properties may include lateral flexibility, pushability, or kink resistance. The one-way valve may be coupled to the device by a tube extending between the one-way valve and the hemostatic valve. The port may be coupled to the device by a tube extending between the port and the hemostatic valve. The sheath may be steerable. The sheath may include an elastic portion such that the sheath is diametrically expandable. The sheath may include a wavy wall such that the sheath is diametrically expandable. The sheath may include an overlapping wall portion such that the sheath is diametrically expandable.

In another implementation, a method of performing a medical procedure using a CBOT device includes: inserting a guidewire through a contralateral femoral access site and crossing over the guidewire to a femoral artery that will receive a large bore delivery sheath; deploying the CBOT device over the guidewire and crossing over a balloon of the CBOT device into an iliac artery that will receive the large bore delivery sheath; injecting contrast media through a lumen of the CBOT device; visualizing, using fluoroscopy, the femoral artery; creating a femoral access site in the femoral artery; preinstalling closure sutures at the femoral access site; installing the large bore delivery sheath through the femoral access site and into the femoral artery; withdrawing the CBOT device into a contralateral iliac artery; advancing the large bore delivery sheath and performing the medical procedure using the large bore delivery sheath; retracting the large bore delivery sheath to the iliac artery; advancing the balloon into the iliac artery; inflating the balloon to occlude blood flow in the iliac artery; pulling back the large bore delivery sheath to the femoral access site; injecting contrast media through the lumen of the CBOT device; visualizing, using fluoroscopy, the iliac and femoral arteries to inspect the iliac and femoral arteries for damage; removing the large bore delivery sheath from the femoral access site; closing the femoral access site; injecting contrast media through the lumen of the CBOT device; visualizing, using fluoroscopy, the access site; withdrawing the CBOT device and the guidewire from the contralateral femoral access site; and closing the contralateral femoral access site.

Such a method of performing a medical procedure using a CBOT device may optionally include one or more of the following features. The CBOT device may comprise: an elongate sheath defining a first lumen and a second lumen; the balloon attached to a distal end portion of the sheath; a hemostatic valve attached to a proximal end portion of the sheath; a one-way valve coupled to the device and configured such that the first lumen is fluidly accessible via the one-way valve; and a port coupled to the device and configured such that the second lumen is fluidly accessible via the port. The balloon may be in fluid communication with the second lumen. A seal of the hemostatic valve may define a proximal end of the first lumen such that the first lumen is accessible via the seal. The elongate sheath may be configured to be diametrically expandable. The method may further comprise deploying a stent device via the lumen of the CBOT device to a portion of the iliac artery or femoral artery. The method may further comprise advancing the balloon to near the femoral access site, and inflating the balloon to provide tamponade for the femoral access site. The medical procedure may be a TAVR procedure. The medical procedure may be one of a procedure to delivery biologics to a heart, a myocardial biopsy procedure, and a pulmonary vascular procedure.

Particular embodiments of the subject matter described in this document are designed to realize one or more of the following advantages. First, in some embodiments the CBOT devices provided herein are singular devices that may replace multiple devices that are currently used to perform the crossover technique. For example, in some cases the crossover technique may currently include the usage of some or all of the following devices: a flexible guidewire, a stiff guidewire, a pigtail diagnostic catheter, a guide catheter, a guide sheath, and multiple peripheral balloons. In contrast, the CBOT devices provided herein are singular multifunction devices that can perform the functions of several of the aforementioned devices in the context of the crossover technique.

Second, the CBOT devices provided herein are specially designed for performance of the crossover technique. In contrast, currently the crossover technique is typically performed using a combination of available devices that are not specifically designed for performance of the crossover technique. Hence, the devices typically used are not optimized for the crossover technique. For example, in some embodiments the CBOT devices provided herein include an elongate compliant balloon that is adaptable for use in a range of vessel sizes. In contrast, currently two or more balloon devices of different sizes are typically used to occlude the common iliac and the femoral arteries respectively.

Third, in some embodiments the CBOT devices provided herein are configured to conveniently allow a covered stent to be deployed via a lumen of the CBOT device. It may be desirable to deploy the covered stent in some circumstances, such as when an iliac or femoral artery has been damaged. The covered stent deployed by the CBOT devices can be implanted to mitigate hemorrhaging of the damaged artery.

Fourth, in some embodiments the CBOT devices provided herein are configured with an expandable sheath. In such embodiments, the outer diameter of the expandable sheath can be advantageously minimized, while maintaining the ability to deploy a device, such as a covered stent, therethrough.

Fifth, in some embodiments the CBOT devices provided herein are configured to deliver a contrast media to the vasculature of the patient. Such a feature can be convenient for visualizing the patient's vasculature and for inspecting for dissections and perforations, for example.

Sixth, in some embodiments the CBOT devices provided herein are configured to be steerable. In some cases, the steerable CBOT devices can be readily navigated through the patient's vasculature, including through tight bends such as where the aorta bifurcates to the iliac arteries, tortuous vasculatures, and the like.

Seventh, in some embodiments the CBOT devices provided herein are configured with different outer diameters and/or different stiffnesses at two or more regions along the length of the devices. Selective usage of such features provides the necessary flexibility, column strength, size, and the like, at various portions to enhance the performance of the CBOT devices.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic representation of a CBOT device in use during a femoral access procedure.

FIG. 2 shows an example CBOT device in accordance with some embodiments.

FIG. 3 shows another example CBOT device in accordance with some embodiments.

FIG. 4 shows another example CBOT device in accordance with some embodiments.

FIG. 5 shows another example CBOT device in accordance with some embodiments.

FIG. 6A is an example cross-sectional view of an expandable sheath in accordance with some embodiments.

FIG. 6B is another example cross-sectional view of an expandable sheath in accordance with some embodiments.

FIG. 6C is another example cross-sectional view of an expandable sheath in accordance with some embodiments.

FIGS. 7A and 7B are a flowchart depicting the performance of a medical procedure including a crossover technique using a CBOT device in accordance with some embodiments.

Like reference numbers represent corresponding parts throughout.

### DETAILED DESCRIPTION

The CBOT devices provided herein include features to improve the crossover balloon occlusion technique used during artery access procedures such as, but not limited to, femoral access procedures used for TAVR procedures, procedures to deliver biologics to a heart, a myocardial biopsy procedure, a pulmonary vascular procedure, and others. The devices include an elongate compliant balloon incorporated with a highly flexible and lubricous sheath system. The balloon is capable of safely occluding from the common iliac artery and down to an access site in the femoral artery. The devices can simplify the CBOT technique and replace multiple devices currently used for the procedure.

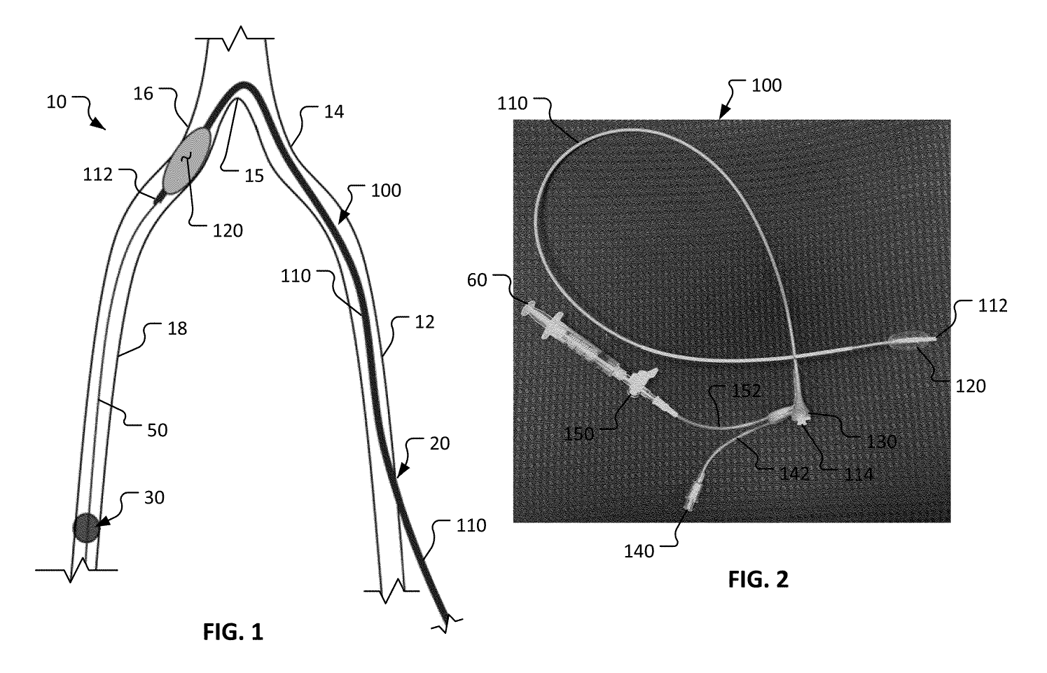

Referring to FIG. 1, a CBOT device 100 can be used to manage a femoral access site 30 in a patient's vasculature 10. Femoral access site 30 may be utilized, for example, as the access site for inserting a large bore delivery sheath system. Such a system may be used for a TAVR procedure, for example, and may require an arteriotomy of greater than 14 Fr at access site 30.

It should be understood that while the CBOT devices provided herein are described in the context of a crossover technique for a TAVR procedure, the CBOT devices are not limited to such a scope. For example, the CBOT devices provided herein can be used in procedures such as, but not limited to, percutaneous aneurysm repair (e.g., PEVAR, EVAR, and TEVAR), leadless pacing delivery procedures, and the like.

To facilitate the placement of CBOT device 100 as shown, in some examples a clinician operator first percutaneously inserts a guidewire 50 at a contralateral access site 20 using an introducer sheath (not shown). The guidewire 50 can be a steerable guidewire in some examples. An imaging modality (e.g., x-ray fluoroscopy) can be used to help the clinician operator navigate guidewire 50 within vasculature 10. The clinician operator pushes guidewire 50 through access site 20 into a contralateral femoral artery 12 and then into a contralateral common iliac artery 14. The clinician operator continues navigating guidewire 50 over an iliac arch 15, into an iliac artery 16 and into a femoral artery 18 towards access site 30. After placement of guidewire 50, the clinician operator can install CBOT device 100 onto guidewire 50 and advance CBOT device 100 over guidewire 50 to the orientation shown.

Referring now to FIGS. 1 and 2, example CBOT device 100 includes a sheath 110, a compliant balloon 120, a hemostatic valve 130, a one-way valve 140, and a stopcock valve 150. Compliant balloon 120 is attached to a distal end portion of sheath 110. A proximal end portion of sheath 110 is attached to hemostatic valve 130. One-way valve 140 is coupled to hemostatic valve 130 via a tube 142. Stopcock valve 150 is coupled to hemostatic valve 130 via a tube 152. Hemostatic valve 130 can include a leak-proof seal through which guidewire 50 and/or other various devices can be installed.

Sheath 110 is a highly flexible elongate tubular construct that can have lubricious properties on the inner and outer diameters of sheath 110. In some embodiments sheath 110 can be made from polymeric materials such as, but not limited to, polytetrafluoroethylene (PTFE), fluorinated ethylene propylene (FEP), Hytrel®, nylon, Picoflex®, Pebax®, and the like. In some embodiments, a coating or surface treatment may be included on sheath 110 to enhance lubricity or other properties.

In the depicted embodiment, the outer diameter of sheath 110 is about 9 Fr, and the inner diameter of sheath 110 is about 7 Fr. However, in some embodiments the outer diameter of sheath 110 is about 5 Fr to about 7 Fr, or about 6 Fr to about 8 Fr, or about 7 Fr to about 9 Fr, or about 8 Fr to about 10 Fr, or about 9 Fr to about 11 Fr, or about 10 Fr to about 12 Fr, or larger than about 12 Fr. In some embodiments, the length of sheath 110 is about 50 cm. However, in some embodiments the length of sheath 110 is about 30 cm to about 60 cm, or about 50 cm to about 80 cm, or about 70 cm to about 100 cm, or about 90 cm to about 120 cm, or greater than about 120 cm.

Sheath 110 defines one or more lumens therethrough. For example, a first lumen is defined by sheath 110, and the first lumen can slidably receive guidewire 50. As described above, CBOT device 100 can be installed into vasculature 10 over guidewire 50. More precisely stated, the first lumen of CBOT device 100 can be slidably engaged with guidewire 50 so that CBOT device 100 can be navigated within vasculature 10 using guidewire 50 to define the path. Accordingly, the first lumen has a distal end opening 112 defined at the distal tip portion of sheath 110. Additionally, the first lumen has a proximal end opening 114 defined at hemostatic valve 130. When CBOT device 100 is installed onto guidewire 50, guidewire 50 is passed into the first lumen of sheath 110 through distal end opening 112 and out proximal end opening 114. In that arrangement, CBOT device 100 can be advanced or withdrawn within arteries 12, 14, 16, and 18 in cooperation with preplaced guidewire 50.

The first lumen of CBOT device 100 can also be used for other purposes. For example, in some implementations the first lumen of CBOT device 100 is used as a conduit for injecting contrast media into vasculature 10. X-ray fluoroscopy can be used to view the flow of the contrast media after it is injected into vasculature 10. In particular examples, contrast media can be injected via CBOT device 100 after the deployment of CBOT device 100 in vasculature 10, but prior to the creation of access site 30. Such a technique may be beneficial for various reasons. For example, the technique can help the clinician operator ascertain a proper puncture point for access site 30, and to visualize the anatomies of the arteries 16 and 18. Such information may help facilitate the performance of a safe and effective TAVR deployment procedure. Additionally, after the procedure including after closing access site 30, contrast media can be injected into vasculature 10 via CBOT device 100 to inspect for leaks in vasculature 10.

In the depicted embodiment, contrast media can be injected via one-way valve 140. One-way valve 140 is normally closed so as to prevent blood flow out of one-way valve 140 when CBOT device 100 in deployed in vasculature 10. One-way valve 140 is coupled to tube 142, and tube 142 is coupled to hemostatic valve 130. The lumen of tube 142 is thereby confluent with the first lumen of sheath 110. As a result, an injection of contrast media into one-way valve 140 will result in passage of the contrast media through tube 142, through the first lumen of sheath 110, and out distal end opening 112 at the distal tip portion of sheath 110.

In some implementations, the first lumen of CBOT device 100 can also be used to measure blood pressure within vasculature 10. As previously described, distal end opening 112 is in fluid communication with one-way valve 140. Therefore, a pressure detection device can be coupled to one-way valve 140 to measure the pressure of the blood at distal end opening 112.

The first lumen of CBOT device 100 can also be used to deploy other devices therethrough. For example, a covered stent device can be deployed through the first lumen of CBOT device 100 to repair a damaged artery, such as if iliac artery 16 or femoral artery 18 become damaged during a TAVR deployment procedure. As will be described further below, in some embodiments sheath 110 is expandable to allow for deployment of devices such as a covered stent while otherwise having a smaller sized outer diameter.

In some embodiments, one or more radiopaque (RO) markers are included on sheath 110. For example, in some embodiments a RO marker is included near the distal end opening 112 and/or at one or more other locations along sheath 110.

Still referring to FIGS. 1 and 2, CBOT device 100 also includes compliant balloon 120 attached to sheath 110. In the depicted embodiment, compliant balloon 120 is disposed near the distal tip portion of sheath 110. In some embodiments, a short portion of sheath 110 protrudes distally from balloon 120. In some embodiments, substantially no portion of sheath 110 protrudes distally from balloon 120.

In some embodiments, balloon 120 is a compliant balloon. That is, balloon 120 expands to a larger size as the pressure within balloon 120 is increased. In other words, the inflated size of balloon 120 can be controlled by the inflation pressure of the inflation media (generally within the range of 0-2 atmospheres). This property of balloon 120 is advantageous in that balloon 120 can be used to safely occlude blood flow at various locations within vasculature 10, including at locations that have a range of different vessel diameters. For that reason, CBOT device 100 can use a single balloon 120 to perform the crossover technique, whereas without CBOT device 100 multiple balloons are typically needed.

Balloon 120 can be constructed of various materials including, but not limited to, polyurethane, polyolefin copolymer, silicone, latex, Gortex®, Kranton®, nylon, Pebax®, PET, and other thermoplastic elastomers. In some embodiments, balloon 120 is about 40 mm long. However, in some embodiments the length of balloon 120 is about 10 mm to about 30 mm, or about 20 mm to about 40 mm, or about 30 mm to about 50 mm, or about 40 mm to about 60 mm, or about 50 mm to about 70 mm, or longer than about 70 mm.

In some embodiments, balloon 120 is configured to be inflated to a diameter of about 1.5 cm when filled with an inflation media at a pressure of about 1 atmospheres to about 2 atmospheres. However, in some embodiments balloon 120 is configured to be inflated to a diameter of about 0.6 cm to about 0.8 cm, or about 0.7 cm to about 0.9 cm, or about 0.8 cm to about 1.0 cm, or about 0.9 cm to about 1.1 cm, or about 1.0 cm to about 1.2 cm, or about 1.1 cm to about 1.3 cm, or about 1.2 cm to about 1.4 cm, or about 1.3 cm to about 1.5 cm, or about 1.4 cm to about 1.6 cm, or about 1.5 cm to about 1.7 cm, or about 1.6 cm to about 1.8 cm, or about 1.7 cm to about 1.9 cm, or greater than 1.9 cm when filled with an inflation media at a pressure of about 1 atmospheres to about 2 atmospheres.

The inflation of balloon 120 can be performed to occlude particular locations of vasculature 10 at various stages during performance of the crossover technique. Various types of inflation media can be used with CBOT device 100. For example, the types of inflation media can include, but are not limited to, saline, CO2, nitrogen, and the like.

An inflation media source 60 can be coupled to CBOT device 100 at stopcock valve 150. While in this example, inflation media source 60 is depicted as a syringe, other types of inflation media sources can be used, including pressurizing devices that can deliver a regulated pressure of inflation media. Further, other types of valves or couplers can be substituted for stopcock valve 150. Stopcock valve 150 is coupled to tube 152. In some embodiments, tube 152 is coupled to hemostatic valve 130 and confluent with a second lumen of sheath 110. In some such embodiments, the second lumen of sheath 110 is a lumen within the wall of sheath 110. As such, the second lumen is typically smaller in diameter than the first lumen of sheath 110. However, other configurations of the first and second lumens are also contemplated. The second lumen of sheath 110 is in fluid communication with the interior of balloon 120. Therefore, an injection of inflation media from inflation media source 60 will travel through tube 152, through the second lumen of sheath 110, and into the interior of balloon 120. Accordingly, the inflation pressure and size of balloon 120 can be controlled by the injection and removal of inflation media from inflation media source 60.

In some embodiments, one or more RO markers are included on or near balloon 120. For example, in some embodiments a RO marker is included near the proximal end and/or the distal end of balloon 120.

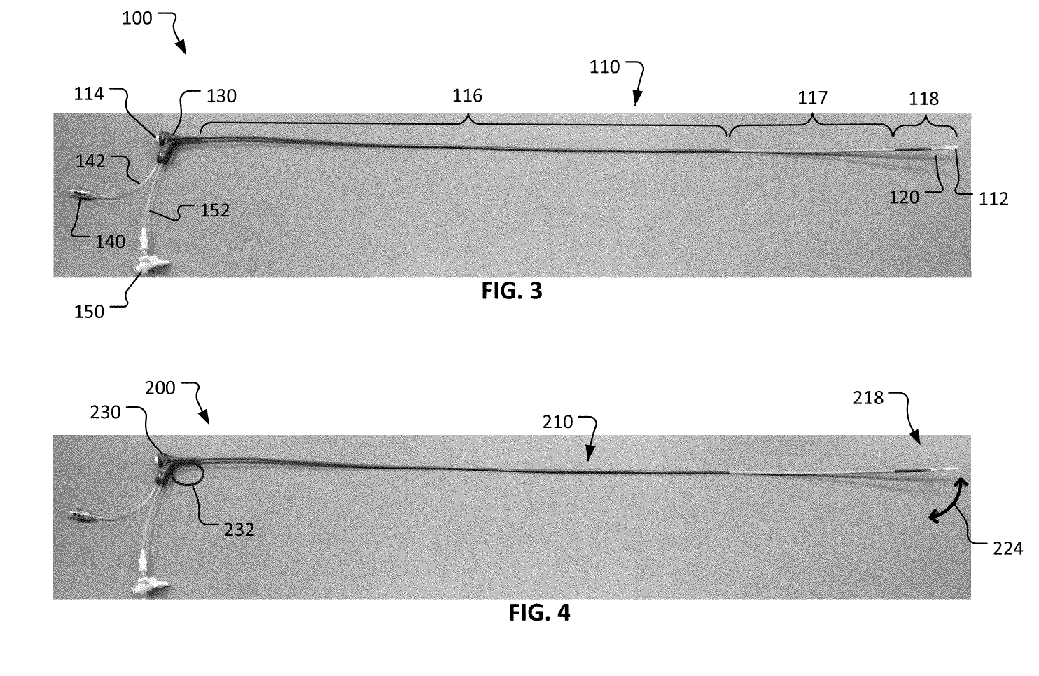

Referring now to FIG. 3, example CBOT device 100 is shown in linear arrangement. It can be seen that, in the depicted embodiment, sheath 110 includes three portions: a proximal portion 116, a middle portion 117, and a distal portion 118. The proximal portion 116, middle portion 117, and distal portion 118 are distinct in that they are constructed differently from each other (as will be described further below). Nevertheless, proximal portion 116, middle portion 117, and distal portion 118 are configured as a singular tubular construct. For example, the first and second lumens of sheath 110 as described above are continuous through each of proximal portion 116, middle portion 117, and distal portion 118.

The differences between proximal portion 116, middle portion 117, and distal portion 118 lie primarily in that they are constructed to have differing mechanical properties such as, but not limited to, lateral flexibilities and column strengths (pushability). Such properties can be selected for each portion 116, 117, and 117 in keeping with the design needs of CBOT device 100. For example, in some embodiments proximal portion 116 is constructed to have a more substantial column strength than portions 117 and 118. The column strength of proximal portion 116 can help facilitate the ability to push CBOT device 100 over guidewire 50 (refer to FIG. 1). The lateral flexibility of proximal portion 116 is less of a design requirement because proximal portion 116 may not need to be contorted as much as portions 117 and 118. That may be the case, for example, because proximal portion 116 may not need to traverse iliac arch 15.

In contrast, in some embodiments middle portion 117 and distal portion 118 are constructed to have more lateral flexibility than proximal portion 116. That may be the case, for example, because middle portion 117 and distal portion 118 need to readily contort so as to traverse iliac arch 15 and/or other tortuous vasculature portions.

The differing properties of proximal portion 116, middle portion 117, and distal portion 118 can be achieved, for example, by using individually differing materials, constructs, reinforcing members, manufacturing methods, and the like, and combinations thereof.

It should be understood that the three distinct portions of sheath 110 (proximal portion 116, middle portion 117, and distal portion 118) are merely one example of how a sheath of a CBOT device provided herein can be constructed. In other CBOT device embodiments envisioned within the scope of this disclosure, the sheath can include a single portion, two portions, four portions, or more than four portions that have differing mechanical properties. In addition, mechanical properties in addition to, or as alternatives to, lateral flexibility and column strength can be selectively determined by the construction of the individual sheath portions. For example, other properties such as, but not limited to, kink resistance, lubricity, durability, torque ability, curve retention, shaft stiffness, full unit tensile, and burst resistance can be selectively determined by the construction of the individual sheath portions.

Referring to FIG. 4, in some embodiments a sheath 210 of a CBOT device 200 can include a steerable tip portion 218. Steerable tip portion 218 can be advantageously included to assist with navigation of the CBOT device 200 over the iliac arch 15 (refer to FIG. 1) and/or through other tortuous portions of vasculature 10.

In some embodiments, a steering actuator 232 is located near a hemostatic valve 230. Steering actuator 232 is manipulatable by the clinician operator to cause inflection of steerable tip portion 218 at various angles as depicted by arrow 224. In some embodiments, a wire interconnects steering actuator 232 and steerable tip portion 218. In some embodiments, the wire is routed through a third lumen located in the wall of sheath 210. However, CBOT device 200 (and the other CBOT device embodiments provided herein) can alternatively be configured in other manners to achieve a steerable sheath 210.

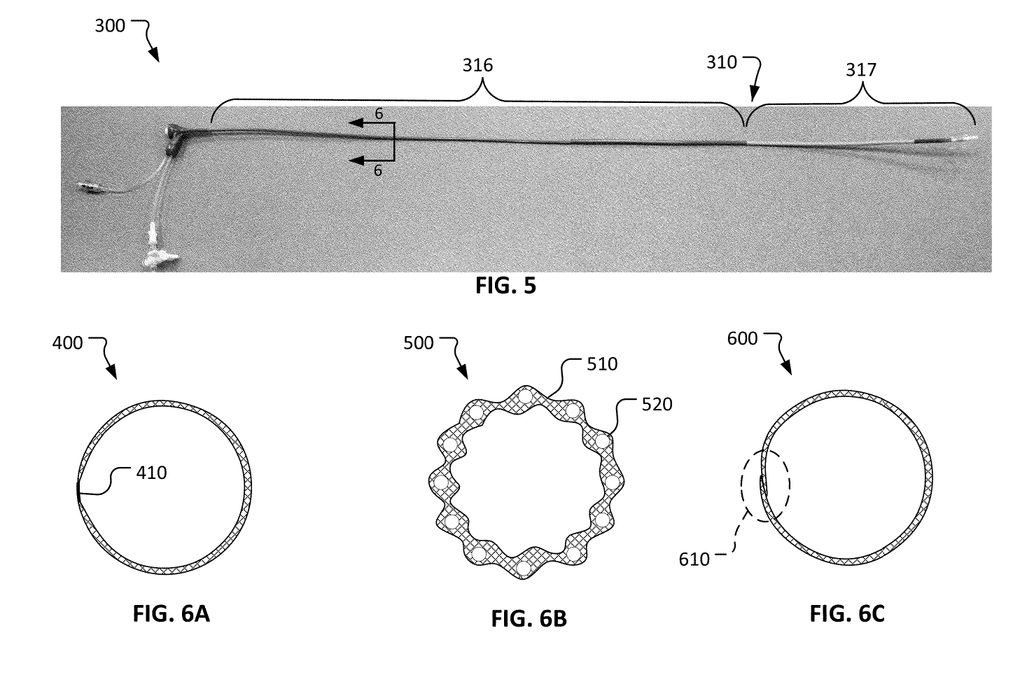

Referring to FIG. 5, in some embodiments a sheath 310 of a CBOT device 300 can be diametrically expandable. In some such embodiments, the entire length sheath 310 is diametrically expandable. Alternatively, in some such embodiments just a proximal portion 316 of sheath 310 is diametrically expandable.

CBOT device 300 with expandable sheath 310 may advantageously allow for a smaller contralateral access site 20 (refer to FIG. 1) in some circumstances. For example, in some embodiments the expandability may allow expandable sheath 310 to be reduced in diameter by 1 to 3 Fr sizes. That is, whereas a conventional sheath may have an inner diameter of 7 Fr and an outer diameter of 9 Fr, an expandable sheath 310 may have an unexpanded inner diameter of 4 Fr, 5 Fr, or 6 Fr and an unexpanded outer diameter of 6 Fr, 7 Fr, or 8 Fr. Nevertheless, an expandable sheath 310 can be expanded to the size of a conventional sheath, e.g., an inner diameter of 7 Fr and an outer diameter of 9 Fr as needed. It should be understood that the aforementioned inner and outer diameters are non-limiting examples, and that lesser and/or greater inner and outer diameters, and expandability amounts, are also envisioned within the scope of this disclosure.

The expandability of expandable sheath 310 may advantageously allow for the deployment of relatively larger medical devices (e.g., stents, etc.) through the lumen of expandable sheath 310, while facilitating a reduced size of contralateral access site 20 (refer to FIG. 1). In some embodiments, expandable sheath 310 retracts to its unexpanded size after being expanded. In some embodiments, expandable sheath 310 merely remains in its expanded size after being expanded.

CBOT device 300 embodiments configured with just proximal portion 316 being diametrically expandable may include a distal portion 317 that is diametrically larger than the unexpanded size of expandable proximal portion 316. In one such non-limiting example, the unexpanded inner diameter of expandable proximal portion 316 may be 5 Fr in some embodiments, and the inner diameter of distal portion 317 may be 7 Fr. Having a larger distal portion 317 may advantageously provide distal portion 317 with enhanced kink resistance, whereas kink resistance of proximal portion 316 may be less necessary, relatively speaking.

Referring now also to FIGS. 6A, 6B, and 6C, expandable sheath 310 can be constructed in various manners as depicted by cross-sectional constructions 400, 500, and 600 taken along section 6-6. Cross-sectional constructions 400, 500, and 600 are shown in unexpanded configurations. Cross-sectional constructions 400, 500, and 600 are designed to enlarge while still providing an essentially leak-proof lumen through expandable sheath 310. Such a feature can be advantageous, for example, so that contrast media can be injected through the lumen after expansion has taken place. However, in some embodiments having an expandable sheath 310, a separate lumen may be used for injection of contrast media.

Cross-section 400 includes an elastic joint 410. Elastic joint 410 can expand (e.g., lengthen) as needed in response to a hoop stress exerted on expandable sheath 310, such as from passing a relatively larger medical device therethrough. In some embodiments, two or more elastic joints 410 may be included.

Cross-section 500 includes an expandable wall 510 by virtue of the wavy cross-sectional profile of expandable wall 510. The inner diameter defined by expandable wall 510 can enlarge as needed in response to a hoop stress exerted on expandable sheath 310, such as from passing a relatively larger medical device therethrough. In such cases, the waviness of expandable wall 510 will lessen as the inner diameter enlarges. In some embodiments, one or more reinforcing ribs 520 may be included to provide, for example, enhanced kink resistance of expandable wall 510.

Cross-sectional profile 600 includes an overlapping wall region 610. The inner diameter defined by cross-sectional profile 600 can enlarge as needed in response to a hoop stress exerted on expandable sheath 310, such as from passing a relatively larger medical device therethrough. In such cases, the amount of overlap of overlapping wall region 610 will lessen as the inner diameter enlarges. An essentially leak-proof conduit can be maintained as cross-sectional profile 600 enlarges.

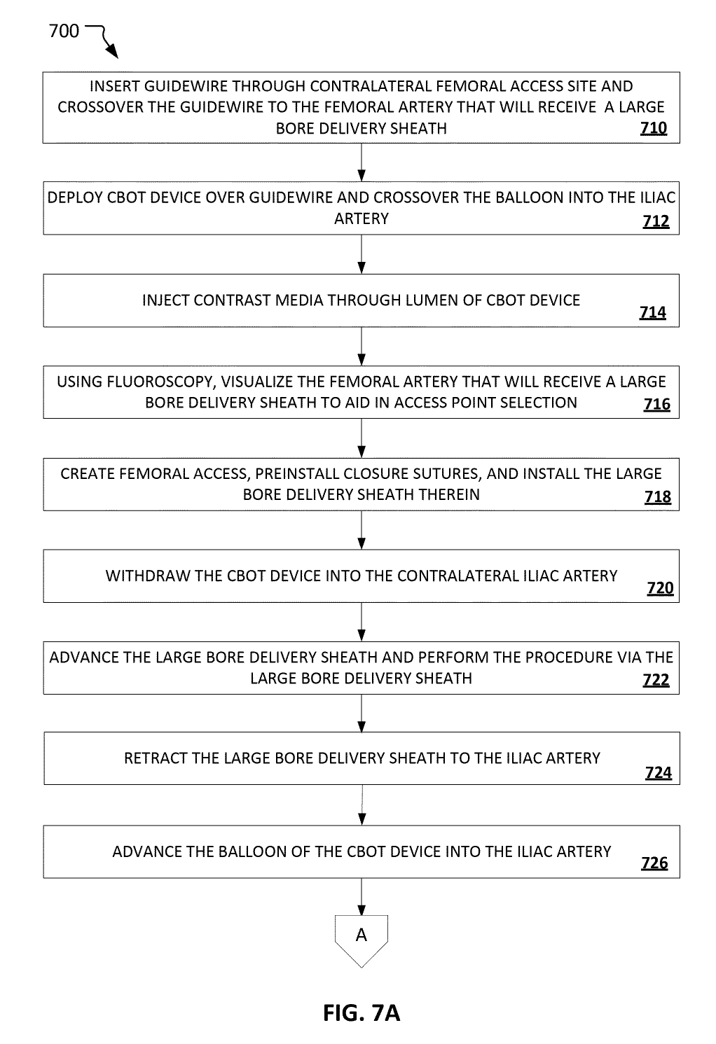

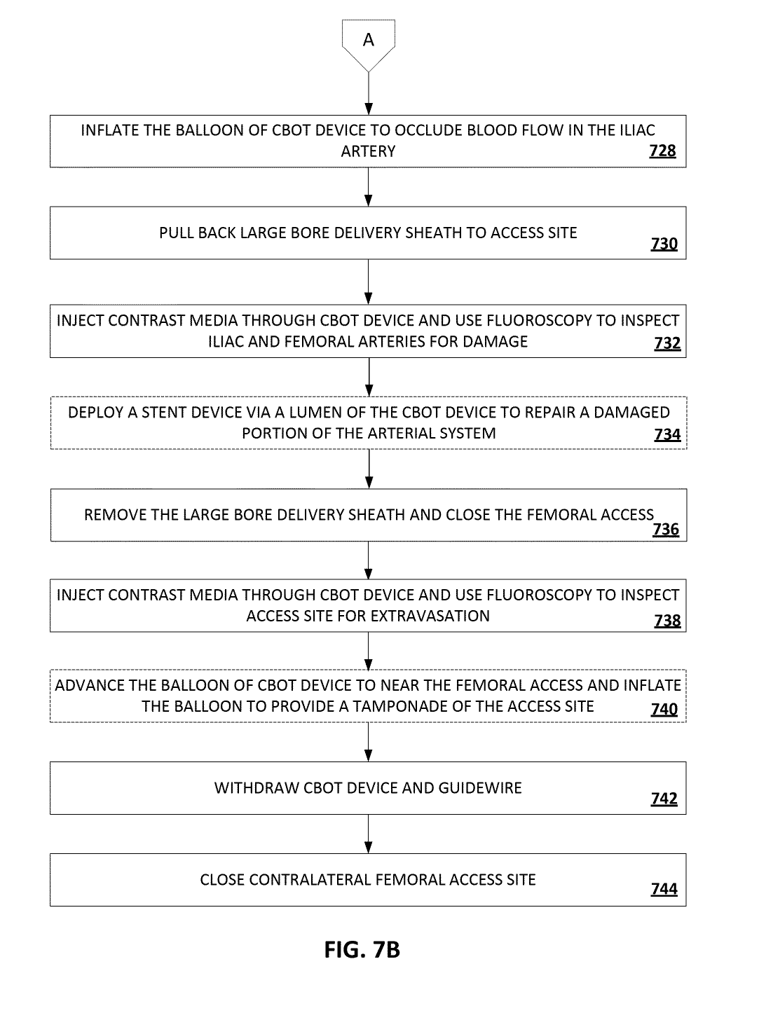

Referring to FIGS. 7A and 7B, a flowchart of an example method 700 of using the CBOT devices provided herein depicts the performance of a medical procedure including a crossover technique using the CBOT device. The CBOT devices provided herein are singular devices that, in the context or method 700 for example, may replace multiple devices that are currently needed to perform the crossover technique. For example, in some current examples the performance of the crossover technique may include the usage of some or all of the following devices: a flexible guidewire, a stiff guidewire, a pigtail diagnostic catheter, a guide catheter, a guide sheath, and two peripheral balloons. In contrast, the CBOT devices provided herein are singular multifunction devices that can perform the functions of several of the aforementioned devices in the context of the crossover technique, as illustrated by method 700.

At step 710, a guidewire is inserted through a contralateral femoral access site and the guidewire is crossed over to the femoral artery that will receive a large bore delivery sheath. The large bore delivery sheath may be a sheath used for a TAVR procedure, for example.

At step 712, a CBOT device as provided herein is deployed over the guidewire and the balloon of the CBOT device is crossed over into the iliac artery that will receive a large bore delivery sheath.

At step 714, contrast media is injected through a lumen of the CBOT device.

At step 716, using fluoroscopy, the femoral artery that will receive the large bore delivery sheath is visualized. The visualization can be used to aid in access point selection.

At step 718, a femoral access site is created, closure sutures are preinstalled, and the large bore delivery sheath is installed in the femoral access site.

At step 720, the CBOT device is withdrawn into the contralateral iliac artery.

At step 722, the large bore delivery sheath is advanced to a target site within the patient, and the procedure (e.g., TAVR deployment procedure) is performed via the large bore delivery sheath.

At step 724, the large bore delivery sheath is retracted to the iliac artery.

At step 726, the balloon of the CBOT device is advanced into the iliac artery.

At step 728, the balloon is inflated to occlude blood flow in the iliac artery.

At step 730, the large bore delivery sheath is pulled back to femoral access site.

At step 732, contrast media is injected via the CBOT device and fluoroscopy is used to inspect iliac and femoral arteries for damage.

At step 734, optionally a stent device can be deployed via a lumen of the CBOT device to repair a damaged portion of the arterial system.

At step 736, the large bore delivery sheath is removed and the femoral access site is closed.

At step 738, contrast media is inject through the CBOT device, and fluoroscopy is used to inspect the access site for extravasation.

At step 740, optionally the balloon of the CBOT device is advanced to near the femoral access site and inflated to provide a tamponade of the femoral access site.

At step 742, the CBOT device and the guidewire are withdrawn from the patient.

At step 744, the contralateral femoral access site is closed. This completes the example method 700 of using the CBOT devices provided herein.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A multifunctional occlusion crossover device comprising:
an elongate sheath defining a first lumen and a second lumen, the elongate sheath being diametrically expandable;
a balloon attached to a distal end portion of the sheath, the balloon in fluid communication with the second lumen;
a hemostatic valve attached to a proximal end portion of the sheath, a seal of the hemostatic valve defining a proximal end of the first lumen such that the first lumen is accessible via the seal;
a one-way valve coupled to the device and configured such that the first lumen is fluidly accessible via the one-way valve; and
a port coupled to the device and configured such that the second lumen is fluidly accessible via the port.

2. The device of claim 1, wherein the elongate sheath includes two or more portions having differing mechanical properties.

3. The device of claim 2, wherein the mechanical properties include lateral flexibility, pushability, or kink resistance.

4. The device of claim 1, wherein the one-way valve is coupled to the device by a tube extending between the one-way valve and the hemostatic valve.

5. The device of claim 1, wherein the port is coupled to the device by a tube extending between the port and the hemostatic valve.

6. The device of claim 1, wherein the sheath is steerable.

7. The device of claim 1, wherein the sheath includes an elastic portion such that the sheath is diametrically expandable.

8. The device of claim 1, wherein the sheath includes a wavy wall such that the sheath is diametrically expandable.

9. The device of claim 1, wherein the sheath includes an overlapping wall portion such that the sheath is diametrically expandable.

10. A method of performing a medical procedure using a CBOT device, the method comprising:
inserting a guidewire through a contralateral femoral access site and crossing over the guidewire to a femoral artery that will receive a large bore delivery sheath;
deploying the CBOT device over the guidewire and crossing over a balloon of the CBOT device into an iliac artery that will receive the large bore delivery sheath;
injecting contrast media through a lumen of the CBOT device;
visualizing, using fluoroscopy, the femoral artery;
creating a femoral access site in the femoral artery;
preinstalling closure sutures at the femoral access site;
installing the large bore delivery sheath through the femoral access site and into the femoral artery;
withdrawing the CBOT device into a contralateral iliac artery;
advancing the large bore delivery sheath and performing the medical procedure using the large bore delivery sheath;
retracting the large bore delivery sheath to the iliac artery;
advancing the balloon into the iliac artery;
inflating the balloon to occlude blood flow in the iliac artery;
pulling back the large bore delivery sheath to the femoral access site;
injecting contrast media through the lumen of the CBOT device;
visualizing, using fluoroscopy, the iliac and femoral arteries to inspect the iliac and femoral arteries for damage;
removing the large bore delivery sheath from the femoral access site;
closing the femoral access site;
injecting contrast media through the lumen of the CBOT device;
visualizing, using fluoroscopy, the access site;
withdrawing the CBOT device and the guidewire from the contralateral femoral access site; and
closing the contralateral femoral access site.

11. The method of claim 10, wherein the CBOT device comprises:
an elongate sheath defining a first lumen and a second lumen;
the balloon attached to a distal end portion of the sheath, the balloon in fluid communication with the second lumen;
a hemostatic valve attached to a proximal end portion of the sheath, a seal of the hemostatic valve defining a proximal end of the first lumen such that the first lumen is accessible via the seal;
a one-way valve coupled to the device and configured such that the first lumen is fluidly accessible via the one-way valve; and
a port coupled to the device and configured such that the second lumen is fluidly accessible via the port.

12. The method of claim 11, wherein the elongate sheath is configured to be diametrically expandable.

13. The method of claim 10, further comprising deploying a stent device via the lumen of the CBOT device to a portion of the iliac artery or femoral artery.

14. The method of claim 10, further comprising advancing the balloon to near the femoral access site and inflating the balloon to provide tamponade for the femoral access site.

15. The method of claim 10, wherein the medical procedure is a TAVR procedure.

16. The method of claim 10, wherein the medical procedure is one of a procedure to delivery biologics to a heart, a myocardial biopsy procedure, and a pulmonary vascular procedure.
